(19) United States
(12) Patent Application Publication
(10) Pub. No.: US 2026/0355209 A1
(43) Pub. Date: Nov. 12, 2026

(54) STABLE READY TO USE COMPOSITION AND USE OF AN IMMUNOSUPPRESIVE AGENT

(71) Applicant: APDM Pharmaceuticals Pvt Ltd, Ahmedabad (IN)

(72) Inventors: Jayanta Kumar Mandal, Ahmedabad (IN); Deep Patel, Ahmedabad (IN); Malay Patel, Ahmedabad (IN)

(21) Appl. No.: 19/671,480

(22) Filed: May 8, 2026

(30) Foreign Application Priority Data
May 9, 2025 (IN) 202521044956

(51) Int. Cl. A61K 31/5377; A61K 9/00; A61K 31/343; A61K 47/02; A61K 47/12; A61K 47/18; A61K 47/20; A61K 47/26

(52) U.S. Cl. CPC A61K 31/5377; A61K 9/0019; A61K 31/343; A61K 47/02; A61K 47/12; A61K 47/183; A61K 47/20; A61K 47/26

(57) ABSTRACT

The present invention relates to a stable, ready-to-use parenteral composition of Mycophenolate and salt thereof. The formulation includes Mycophenolate Mofetil as the active pharmaceutical ingredient (API), optimized with excipients for solubilization, pH adjustment, and tonicity balance. Solubilizers are used to enhance the stability of the API in an aqueous medium. The formulation also includes formation of Mycophenolic acid as one more active pharmaceutical ingredient to provide synergistic effect. The formulation maintains an acidic pH, adjusted using sodium hydroxide and hydrochloric acid, ensuring optimal stability and bioavailability. Water for injection serves as the vehicle, completing the formulation. The present invention provides a user-friendly, cost-effective solution for clinical applications requiring Mycophenolate Mofetil in injectable form.

### RELATED APPLICATIONS

This application claims priority to Indian Application No. 202521044956 filed May 9, 2025 which is incorporated herein by reference in its entirety.

### FIELD OF THE INVENTION

The present invention relates to the pharmaceutical field, specifically to a ready-to-use parenteral composition of Mycophenolate and its pharmaceutically acceptable salts. The invention focuses on an injectable formulation containing Mycophenolic acid or its salts as the active ingredient, along with carefully selected excipients to enhance solubility, stability, and patient safety. Mycophenolic acid is also generated during storage of the formulation which also works as an additional active ingredient providing synergistic effect to provide the same effect of that of approved indications of approved dosage regiment. The formulation achieves precise tonicity, optimal pH, and consistent sterility, making it suitable for clinical administration. This invention addresses the challenges of accurate reconstitution, aseptic technique for the same, storage stability, preparation convenience, and therapeutic efficacy associated with marketed Mycophenolate parenteral formulations, providing an advanced composition for use in the treatment of medical conditions requiring immunosuppressive therapy.

### BACKGROUND OF THE INVENTION

The present invention relates to the pharmaceutical field, particularly to a ready-to-use parenteral composition of Mycophenolic acid and its pharmaceutically acceptable salts. Mycophenolate mofetil (MMF) is a prodrug of mycophenolic acid (MPA), an immunosuppressive agent widely used in clinical practice to prevent organ rejection in transplant patients and treat autoimmune disorders. It acts by inhibiting inosine monophosphate dehydrogenase (IMPDH), a key enzyme in the de novo synthesis of guanine nucleotides. This mechanism selectively targets the proliferation of T and B lymphocytes, which are critical to immune response, making MMF an essential component of immunosuppressive regimens.

Mycophenolate mofetil is primarily used in organ transplantation, including kidney, heart, and liver transplants, to prevent rejection. It is also employed in the treatment of autoimmune diseases such as systemic lupus erythematosus (SLE), rheumatoid arthritis, and psoriasis. MMF has shown efficacy in off-label uses for treating various inflammatory and dermatological conditions. The availability of a ready-to-use injectable formulation expands its application in clinical settings, providing an alternative for patients who cannot tolerate oral medications.

Mycophenolate mofetil has been extensively used in oral formulations for its ability to achieve consistent bioavailability and therapeutic efficacy. However, certain clinical conditions, such as gastrointestinal intolerance, critical illness, or the inability to administer oral formulations due to surgical interventions, necessitate the use of a parenteral form.

U.S. Pat. No. 4,753,935 entitled “Morpholinoethyl Esters of Mycophenolic Acid and Pharmaceutical Compositions” discloses Mycophenolate Mofetil, the morpholinoethyl ester of mycophenolic acid, and certain simple ester derivatives thereof.

U.S. Pat. No. 4,808,592 entitled “Method of Treating Diseases by Administering Morpholinoethyl Ester of Mycophenolic Acid and Derivatives Thereof” discloses a method of treating inflammatory and psoriatic diseases in mammals by administering a therapeutically effective amount of mycophenolate mofetil, the morpholinoethyl ester of mycophenolic acid, or certain simple ester derivative thereof.

PCT Patent Application No. WO1995/07902 discloses a crystalline anhydrous compound of mycophenolate mofetil, complexed as a salt with an anion from a specified group, offering enhanced solubility and stability. This compound is utilized in intravenous formulations for treating autoimmune disorders and allograft rejection, providing significant pharmacokinetic advantages over existing forms.

U.S. Pat. No. 5,543,408 relates to a crystalline anhydrous salt of mycophenolate mofetil complexed with specific anions, providing a stable and effective pharmaceutical formulation. This invention offers advantages in solubility and stability, facilitating the preparation of intravenous formulations that maintain therapeutic efficacy for treating autoimmune disorders and inflammatory diseases.

PCT Patent Application No. WO2004/087174 claims a pharmaceutical composition in powder or lyophilized form for parenteral administration containing mycophenolic acid (MPA), a salt, or a prodrug thereof. This composition is applicable in clinical settings for preventing or treating organ transplant rejection and managing immune-mediated diseases.

PCT Patent Application No. WO2011/061761 provides a pharmaceutical composition for parenteral administration that includes mycophenolate mofetil or its pharmaceutically acceptable salts in amorphous form. The invention has drawbacks of unstable cake during lyophilization.

Chinese Patent No. CN106913531 describes mycophenolate mofetil freeze-dried composition that includes mycophenolate mofetil, a stabilizer (citric acid), a cosolvent (sodium benzoate), a pH regulator, and water for injection. The resulting composition exhibits good re-solubility and maintains its quality over time, making it more suitable for clinical use.

Chinese Patent No. CN106943359 discloses a freeze-dried composition of mycophenolate mofetil prepared with citric acid, polysorbate 80, disodium tetraborate, a pH regulator, and water for injection, characterized by a specific weight ratio of mycophenolate mofetil to disodium tetraborate (25:3-5) and a pH adjustment using hydrochloric acid.

Chinese Patent No. CN108272755 provides a preparation method for mycophenolate mofetil hydrochloride freeze-dried for injection, characterized by specific raw materials and their weight ratios, including mycophenolate mofetil, polysorbate 80, anhydrous citric acid, hydrochloric acid, anhydrous ethanol, and sodium hydroxide, with defined procedural steps for mixing, pH adjustment, and freeze-drying.

As per above prior-arts, the inventors of the present invention found that the development of injectable formulations that ensure precise and controlled drug delivery. While intravenous formulations of MMF are available, they often require reconstitution prior to administration followed by dilution, leading to potential errors in preparation, loss of sterility, and inconvenience for healthcare providers.

Existing injectable formulations of MMF are typically lyophilized powders, requiring reconstitution with a suitable diluent before use. These formulations introduce the risk of contamination and variability in the final preparation.

Additionally, improper handling during reconstitution can compromise the stability of the active ingredient. Therefore, the inventors of the present invention realized the need for a ready-to-use injectable formulation that eliminates these risks while maintaining the drug's stability, efficacy, and safety.

Ready-to-use parenteral formulations offer significant advantages over traditional lyophilized forms. They eliminate the need for reconstitution, reducing preparation time and minimizing the risk of contamination. These formulations enhance patient safety and convenience for healthcare providers. Additionally, ready-to-use formulations improve compliance with good manufacturing practices (GMP) by ensuring consistent quality and sterility.

The present invention addresses the need for a ready-to-use parenteral formulation by utilizing a specific combination of excipients that provide stability, solubility, and sterility. Sodium chloride is incorporated to achieve isotonicity, ensuring compatibility with physiological conditions.

pH adjustment is critical to maintaining the stability of MMF in aqueous solutions. Hydrochloric acid and sodium hydroxide are employed to adjust the pH to an optimal value of 3.0, which prevents degradation and ensures the formation of a stable solution. Water for injection serves as the vehicle, providing the medium for the active pharmaceutical ingredient and excipients to dissolve and maintain homogeneity.

Surprisingly, the inventors of the present invention found that during storage, Mycophenolic acid, the active metabolite of Mycophenolate mofetil, is generated in the formulation.

This phenomenon enhances the therapeutic efficacy of the product by providing a synergistic effect, leveraging the pharmacological activity of both the prodrug and its metabolite. Stability profile of the formulation ensures that the generation of Mycophenolic acid remains within acceptable limits, maintaining the overall safety and efficacy of the product.

The invention further optimizes the manufacturing process to ensure reproducibility and scalability. The pH is adjusted to maintain stability, and the solution is filtered through a 0.2 μm filter to ensure sterility.

The present invention combines innovative formulation design with advanced manufacturing techniques to deliver a ready-to-use injectable product that meets these objectives. This advancement has the potential to significantly improve patient care and expand the clinical utility of Mycophenolate mofetil in various therapeutic areas.

### OBJECTIVE OF THE INVENTION

The primary objective of the present invention is to provide a ready-to-use parenteral composition of mycophenolate or its salts that offers enhanced stability, efficacy, and convenience for medical use, ensuring accurate dosing and ease of administration.

One objective of the present invention is to formulate a stable composition of mycophenolate mofetil or its salts or acid form that remains in a ready-to-use injectable form, eliminating the need for reconstitution steps and reducing preparation time during critical medical procedures.

One more objective of the present invention is to achieve a composition that maintains the potency and efficacy of mycophenolate mofetil over its shelf life by incorporating suitable excipients and controlling pH and tonicity to minimize degradation.

Yet another objective of the present invention is to develop a parenteral formulation where mycophenolic acid, generated during storage, acts as a synergistic active ingredient to enhance the therapeutic effects of the composition.

An additional objective of the present invention is to utilize a specific combination of buffering agents, including citric acid monohydrate and tri-sodium citrate dihydrate, to maintain the desired pH range, ensuring the chemical stability and solubility of the active pharmaceutical ingredient (API).

One objective of the present invention is to provide a formulation that is isotonic through the inclusion of sodium chloride, making it suitable for intravenous administration without causing adverse effects.

Another objective of the present invention is to ensure the formulation process is efficient and scalable, involving straightforward manufacturing steps, such as adjusting pH, solubilizing excipients, and filtration, to produce a high-quality, reproducible product.

A further objective of the present invention is to offer a ready-to-use injectable composition packaged in a clear tubular glass vial with bromobutyl rubber stoppers and aluminum flip-off seals, ensuring compatibility with the formulation and preventing contamination.

One more objective of the present invention is to design a composition that adheres to regulatory requirements and ensures quality, safety, and effectiveness by leveraging potency corrections for the active ingredient and maintaining strict control over excipient interactions.

### SUMMARY OF THE INVENTION

The present invention relates to a ready-to-use parenteral composition of mycophenolate or its salts or free acid form or their combinations, which provides enhanced stability, ease of use, and therapeutic efficacy. The invention addresses the challenges associated with conventional mycophenolate formulations, including the need for reconstitution, potential degradation, and suboptimal pharmacological outcomes, by developing a stable and effective injectable formulation.

The primary aspect of the present invention is to provide a ready-to-use injectable formulation of mycophenolate mofetil or its salts that eliminates the need for reconstitution prior to administration. This feature ensures immediate availability, reduces preparation time, and minimizes the risk of errors during preparation and administration.

One aspect of the present invention is to offer a parenteral composition that maintains the stability of the active pharmaceutical ingredient (API) by using a carefully selected combination of excipients and optimizing the pH and tonicity that ensures the formulation remains chemically stable throughout its shelf life.

One more aspect of the present invention is to ensure the formulation provides therapeutic consistency by incorporating solubilizer, which enhances the solubility and homogeneity of mycophenolate mofetil in the aqueous vehicle. This ensures a uniform drug concentration in each administered dose, thereby improving patient outcomes.

Yet another aspect of the present invention is to provide a composition that includes tonicity-adjusting agent. This ensures that the formulation is isotonic and suitable for intravenous administration, thereby reducing the risk of adverse osmotic effects and enhancing patient safety during treatment.

One aspect of the present invention is that during storage mycophenolic acid is being available to the formulation which helps to provide fast action to patients.

An additional aspect of the present invention is to develop a manufacturing process that is efficient and scalable, enabling consistent production of the ready-to-use formulation. The process includes steps such as sequential addition of excipients, pH adjustment, and filtration to ensure the final product meets stringent quality standards.

Yet another aspect of the present invention is to simplify the administration of mycophenolate by providing a stable and isotonic injectable composition.

The present invention further aims to provide a parenteral composition that is versatile and adaptable for use with mycophenolate mofetil, mycophenolate mofetil hydrochloride, and mycophenolate sodium, with potency corrections made as needed. This adaptability ensures the formulation meets diverse therapeutic requirements while maintaining consistent quality and efficacy.

The formulation developed in the present invention eliminates the need for stabilizers and achieves stability through precise control of pH, excipient selection, and manufacturing processes.

The invention ensures the ready-to-use injectable composition meets the needs of healthcare providers and patients by offering a stable, effective, and convenient solution for administering mycophenolate mofetil.

This invention is particularly advantageous in clinical settings where immediate availability and ease of administration are critical. By eliminating the need for reconstitution and ensuring a stable, ready-to-use product, the invention addresses key challenges faced by healthcare professionals and patients. During storage mycophenolic acid is being available to the formulation which helps to provide fast action to patients.

### DETAILED DESCRIPTION OF THE INVENTION

The following detailed description of the present subject matter the various embodiments. These embodiments are described in sufficient detail to enable those skilled in the art to practice the present subject matter. Other embodiments may be utilized and changes may be made without departing from the scope of the present subject matter.

References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The term ‘drug’ or ‘active ingredient’ or ‘active pharmaceutical ingredient (API)’ herein refers to Mycophenolate Mofetil 5.88 mg/ml or Mycophenolate Mofetil hydrochloride 6.38 mg eq. to Mycophenolate Mofetil 5.88 mg or Mycophenolate Sodium 4.65 mg eq. to Mycophenolate Mofetil 5.88 mg or Mycophenolic acid eq. 4.35 mg to Mycophenolate Mofetil 5.88 mg.

The present invention relates to a ready-to-use parenteral composition of Mycophenolate and its pharmaceutically acceptable salts, particularly Mycophenolate Mofetil, Mycophenolate Mofetil Hydrochloride, and Mycophenolate Sodium. This invention provides a stable, effective, and easy-to-administer injectable formulation designed to improve the therapeutic delivery of Mycophenolate while addressing challenges such as stability, solubility, and patient compliance.

The primary embodiment of the present invention is to provide a ready-to-use injectable composition comprising Mycophenolate Mofetil as the active pharmaceutical ingredient (API). This composition includes excipients specifically selected to ensure stability, proper tonicity, and a physiologically acceptable pH for parenteral administration.

One more embodiment of the present invention is of generation of Mycophenolic acid during storage, which contributes to the synergistic therapeutic effect to the parenteral formulation of mycophenolate mofetil.

One embodiment of the present invention is to utilize combination of buffering agents to maintain the pH of the solution in acidic range. These buffering agents ensure that the formulation remains stable and prevents degradation of the active ingredient.

One more embodiment of the present invention is the inclusion of sodium chloride for tonicity adjustment. Sodium chloride ensures that the formulation is isotonic with physiological fluids, which is critical for patient safety and comfort during administration. Tonicity adjustment is particularly important in parenteral formulations to avoid irritation or damage to the tissues of patient.

Yet another embodiment of the present invention is the use of solubilizer. Polysorbate 80 is a well-known surfactant used in parenteral formulations to enhance the solubility of hydrophobic drugs. In this invention, it plays a crucial role in maintaining the solubility of Mycophenolate Mofetil, ensuring a uniform and stable solution that is suitable for injection.

One more embodiment of the present invention encompasses the use of hydrochloric acid and sodium hydroxide as pH adjusters. These agents allow precise control of the pH of the solution during manufacturing, ensuring that the final formulation maintains a pH in acidic range, which is about 3-5. This pH range is selected based on the stability profile of Mycophenolate Mofetil and its compatibility with other excipients in the formulation.

Another embodiment of the present invention provides use of vehicle which is water for injection, serving as the primary solvent. The use of water for injection ensures that the formulation meets the stringent quality requirements for parenteral products. Additionally, the formulation process involves the use of filtration through a 0.2 μm filter to achieve sterility and remove any particulate matter.

In yet another embodiment of the present invention provides a detailed manufacturing procedure to ensure reproducibility and quality. The process begins with the preparation of an aqueous solution of citric acid monohydrate and tri-sodium citrate dihydrate, followed by the addition of sodium chloride and Polysorbate 80. The pH of the solution is carefully adjusted using hydrochloric acid and sodium hydroxide before adding Mycophenolate Mofetil. The final solution is filtered to achieve sterility and filled into 100 mL to 200 mL clear glass vials or in intravenous (IV) bags of different material of construction. The vials and/or bags are sealed rubber stoppers and aluminum flip-off seals to maintain the integrity and sterility of the product.

One more embodiment of the present invention is to provide a formulation that ensures a shelf life suitable for commercial distribution and storage under varying environmental conditions. Stability studies have been conducted to confirm the robustness of the formulation under accelerated and long-term storage conditions.

Yet another embodiment of the present invention is the environmental and economic advantages of the ready-to-use formulation. By eliminating the need for reconstitution, the formulation reduces the use of additional materials and resources, such as sterile water for injection and preparation equipment. This feature contributes to cost savings for healthcare providers and minimizes the environmental impact of the product.

One additional embodiment of the present invention is to provide a versatile formulation that accommodates variations in the active ingredient. The formulation can be adapted to include Mycophenolate Mofetil Hydrochloride or Mycophenolate Sodium, with appropriate potency corrections based on the actual assay and water content. This flexibility ensures that the invention covers a broad range of therapeutic needs and regulatory requirements.

Further embodiment of the present invention includes the primary packaging material comprises 100 to 200 mL clear glass vials, rubber stoppers, and aluminum flip-off seals. These components are selected for their compatibility with the formulation and their ability to maintain sterility and stability. Secondary packaging includes white folded cartons for each vial, ensuring protection during transportation and storage.

Yet another embodiment of the present invention is the provision of a composition with pharmaceutically acceptable excipients selected from a range of tonicity modifiers, buffers, solubilizers, pH adjusting agents and solvent for vehicle, or mixtures thereof. These excipients are carefully chosen to enhance the stability, delivery, and effectiveness of the active ingredients, ensuring that the composition remains potent and easy to apply, ultimately improving patient compliance and therapeutic outcomes.

Tonicity modifiers are selected one or more from Sodium chloride, Potassium chloride, mannitol, dextrose or mixtures thereof.

Buffers are selected one or more from Citric acid monohydrate, Tri-sodium citrate dihydrate. Sodium phosphate (monobasic or dibasic), Acetate buffer, Histidine buffer, or mixtures thereof.

Solubilizers are selected one or more from Polysorbate 80, Polysorbate 20, Polyoxyl 35 castor oil, Polyethylene glycol, Propylene glycol, or mixtures thereof.

However, the choice of excipients is limited to those suitable for parenteral formulations to ensure compatibility, safety and compatible with active ingredients of the present invention.

The present invention also addresses the therapeutic advantages of the ready-to-use formulation. The elimination of reconstitution steps reduces the risk of dosing errors and contamination, improving patient safety and compliance. This feature is particularly beneficial in clinical settings where quick and accurate administration of the drug is critical.

The manufacturing process described in the present invention is scalable and suitable for industrial production. The use of standard equipment and processes ensures that the formulation can be produced efficiently and consistently. The process also includes stringent quality control measures, such as in-process pH adjustments and sterility testing, to meet regulatory standards for parenteral products.

One further embodiment of the present invention is to provide a ready-to-use parenteral composition comprising mycophenolate sodium in combination with an osmotic agent selected from dextrose, mannitol, or mixtures thereof. The inventors of the present invention establish that inclusion of such osmotic agents provides isotonicity and improves stability of the formulation during storage.

Another embodiment of the present invention is to provide a formulation comprising an antioxidant selected from monothioglycerol, L-cysteine hydrochloride, or ascorbic acid. The inventors of the present invention establish that incorporation of the antioxidant system reduces oxidative degradation and maintains impurity levels within acceptable limits during long-term storage.

Yet another embodiment of the present invention is to provide a formulation further comprising a chelating agent selected from disodium edetate. The inventors of the present invention establish that the chelating agent enhances stability by reducing metal ion-induced degradation.

One more embodiment of the present invention is to provide a ready-to-dilute formulation having a higher concentration of mycophenolate suitable for dilution prior to administration. The inventors of the present invention establish that such formulation enables flexible dosing and reduces storage volume while maintaining stability.

Another embodiment of the present invention is to provide a formulation demonstrating total impurities below about 1.5% over 12 months under accelerated and long-term storage conditions. The inventors of the present invention establish that the formulation maintains chemical integrity and meets regulatory stability requirements.

A ready-to-use parenteral pharmaceutical composition as per the present invention comprising: mycophenolate or a pharmaceutically acceptable salt or free acid thereof; a buffering system maintaining pH in an acidic range; a tonicity adjusting agent; and an aqueous vehicle; wherein the composition is present as a sterile solution suitable for direct administration without reconstitution and exhibits controlled impurity formation during storage at 25 °C.

The composition as per the present invention, wherein the mycophenolate is selected from mycophenolate mofetil, mycophenolate sodium, mycophenolate mofetil hydrochloride, mycophenolic acid, or combinations thereof.

The composition as per the present invention, wherein the buffering system comprises citric acid, citrate salts, or combinations thereof, maintaining pH in the range of about 2.5 to about 6.5.

The composition as per the present invention, wherein the tonicity adjusting agent is selected from sodium chloride, dextrose, mannitol, or combinations thereof, providing isotonicity suitable for parenteral administration.

The composition as per the present invention, further comprising a solubilizer selected from polysorbate 80, polysorbate 20, polyoxyl castor oil, or combinations thereof, wherein the solubilizer maintains the active ingredient in a dissolved state.

The composition as per the present invention, further comprising an antioxidant selected from monothioglycerol, L-cysteine hydrochloride, ascorbic acid, or combinations thereof, wherein the antioxidant reduces oxidative degradation during storage.

The composition as per the present invention, further comprising a chelating agent selected from disodium edetate, wherein the chelating agent reduces metal ion-induced degradation.

The composition as per the present invention, wherein the composition exhibits a controlled impurity profile characterized in that: (i) total impurities remain below about 1.5% after storage for 12 months under accelerated conditions of about 25 °C and 60% relative humidity; and (ii) individual impurities increase in a gradual and predictable manner without formation of any new major degradation product.

A ready-to-dilute sterile parenteral pharmaceutical composition as per the present invention comprising: (a) mycophenolate or a pharmaceutically acceptable salt or free acid thereof in a in a concentrated form; (b) a buffering system; (c) a solubilizer; and (d) an aqueous vehicle; wherein the composition is provided as a concentrated solution requiring dilution with a pharmaceutically acceptable diluent prior to administration.

A pharmaceutical product as per the present invention comprising: (a) the composition as claimed in any one of claims 1 to 8; and (b) a sterile container selected from a glass vial, infusion bag, or prefilled container system; wherein the composition is maintained in a ready-to-use form without requirement of reconstitution prior to administration.

In summary, the present invention provides a novel and effective solution for delivering Mycophenolate and its salts as a ready-to-use injectable composition. The formulation addresses key challenges such as stability, solubility, and patient compliance, offering significant advantages over existing products. The detailed description and examples provided herein demonstrate the robustness, versatility, and therapeutic potential of the invention, paving the way for its successful implementation in clinical practice.

### Example-1: Mycophenolate Mofetil Injection 500 mg Per Vial (Ready to Use)

| Sr. | | | Role in |
|---|---|---|---|
| No. | Ingredients | mg/mL | Formulation |
| 1 | Mycophenolate mofetil # | 5.882 * | API |
| 2 | Sodium Chloride | 6.50 | Tonicity |
| | | | adjustment |
| 3 | Citric acid monohydrate | 8.61 | Buffer |
| 4 | Tri-sodium citrate dihydrate | 2.65 | Buffer |
| 5 | Polysorbate 80 | 10.00 | Solubilizer |
| 6 | Hydrochloric acid | Q.S. to pH 3.0 | pH adjustment |
| 7 | Sodium hydroxide | Q.S. to pH 3.0 | pH adjustment |
| 8 | Water for injection | Q.S. to 1 mL | Vehicle |

* Potency correction to be done while dispensing considering the actual assay and water content.
# Also consider Mycophenolate mofetil hydrochloride and Mycophenolate Sodium. Potency correction to be done accordingly.

### Process for Preparation:

1. Water for Injection (90% of the total batch size) was taken into an S.S. manufacturing vessel.
2. The specified quantity of Citric acid monohydrate was added and stirred to ensure complete solubilization.
3. The specified quantity of Tri-sodium citrate dihydrate was added and stirred to ensure complete solubilization.
4. The specified quantity of Sodium chloride was added and stirred to ensure complete solubilization.
5. The specified quantity of Polysorbate 80 was added and stirred to ensure complete mixing.
6. The pH of the solution was adjusted to pH 3.0 using Sodium hydroxide and/or Hydrochloric acid solution.
7. The specified quantity of Mycophenolate mofetil was added and stirred to ensure complete solubilization.
8. The pH of the solution was adjusted to pH 3.0 using Sodium hydroxide and/or Hydrochloric acid solution if required.
9. The volume was made up using the remaining quantity of Water for Injection.
10. The bulk solution was filtered through a 0.2 μm filter to get the final formulation.

Following is some information about Packaging material:

PRIMARY PACKAGING MATERIAL

| | |
|---|---|
| 1 | 100 mL Clear Tubular Glass vial and 200 mL PVC IV bags |
| 2 | Bromobutyl Rubber Stopper |
| 3 | Aluminum Flip off Seal |

SECONDARY PACKAGING MATERIAL

| | |
|---|---|
| 4 | White folded carton per vial |

Total Target Fill Volume of final vial with vehicle is 85 mL for Mycophenolate Mofetil 500 mg and 170 mL for Mycophenolate Mofetil 1000 mg.

### Example-2: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-3: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Hydroxypropyl ß-cyclodextrin was added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-4: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 40.0% of Betadex Sulfobutyl Ether Sodium was added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-5: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Polysorbate 80 was added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-6: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Hydroxypropyl ß-cyclodextrin and up to 20.0% of Polysorbate 80 were added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-7: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Polysorbate 80 and up to 40.0% Betadex Sulfobutyl Ether Sodium was added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-8: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Hydroxypropyl ß-cyclodextrin and up to 35.0% propylene glycol were added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-9: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Hydroxypropyl ß-cyclodextrin and up to 60.0% were added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-10: Ready to Use Mycophenolate Mofetil or Mycophenolate Mofetil Hydrochloride or Mycophenolic Acid or Mycophenolate Sodium Injection

Water for Injection (90% of the total batch size) was taken into a stainless steel manufacturing vessel. Up to 2.5% of Citric acid monohydrate, up to 7.0% of Trisodium citrate dihydrate, up to 2.0% Acetic acid and up to 9.0% Sodium acetate were added and stirred to ensure complete solubilization. Then, up to 9.0% of Sodium chloride, up to 60% of Dextrose and up to 60% of Mannitol were added and stirred to ensure complete solubilization and tonicity adjustment. Up to 20.0% of Hydroxypropyl ß-cyclodextrin, up to 20% of polysorbate 80 and up to 35.0% of propylene glycol and up to up to 60.0% polyethylene glycol were added to ensure complete mixing. The pH of the solution was adjusted to pH 3.0 using Sodium hydroxide and/or Hydrochloric acid solution. Active ingredient was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation.

### Example-11: Ready to Use Mycophenolate Mofetil Injection

Water for Injection (90% of the total batch size) was taken into a stainless steel manufacturing vessel. Up to 2.5 mg per ml of Citric acid monohydrate, up to 10 mg per ml of Polysorbate 80 were added to it. Then, up to 9.0 mg per ml of Sodium chloride or up to 60 mg per ml of Dextrose or up to 60 mg per ml of Mannitol were added and stirred to ensure complete solubilization and tonicity adjustment. The pH of the solution was adjusted to pH 2.5 using Sodium hydroxide and/or Hydrochloric or Acetic acid solution. Mycophenolate mofetil about 5.88 mg per ml was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation of 1 ml.

### Example-12: Ready to Use Mycophenolate Mofetil Hydrochloride Injection

Water for Injection (90% of the total batch size) was taken into a stainless steel manufacturing vessel. Up to 2.5 mg per ml of Citric acid monohydrate, up to 10 mg per ml of Polysorbate 80 were added to it. Then, up to 9.0 mg per ml of Sodium chloride or up to 60 mg per ml of Dextrose or up to 60 mg per ml of Mannitol were added and stirred to ensure complete solubilization and tonicity adjustment. The pH of the solution was adjusted to pH 7 to 8 using Sodium hydroxide and/or Hydrochloric or Acetic acid solution. Mycophenolate mofetil hydrochloride about 6.38 mg per ml was added and stirred to ensure complet e solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation of 1 ml.

### Example-13: Ready to Use Mycophenolate Sodium Injection

Water for Injection (90% of the total batch size) was taken into a stainless steel manufacturing vessel. Up to 2.5 mg per ml of Citric acid monohydrate was added to it. Then, up to 9.0 mg per ml of Sodium chloride or up to 60 mg per ml of Dextrose or up to 60 mg per ml of Mannitol were added and stirred to ensure complete solubilization and tonicity adjustment. The pH of the solution was adjusted to pH 2.5 using Sodium hydroxide and/or Hydrochloric or Acetic acid solution. Mycophenolate sodium about 4.65 mg per ml was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation of 1 ml.

### Example-14: Ready to Use Mycophenolic Acid Injection

Water for Injection (90% of the total batch size) was taken into a stainless steel manufacturing vessel. Up to 1 mg per ml of Sodium phosphate monobasic Monohydrate, up to 5 mg per ml of Sodium phosphate dibasic dihydrate and up to 10 mg per ml of Polysorbate 80 were added to it. Then, up to 9.0 mg per ml of Sodium chloride or up to 60 mg per ml of Dextrose or up to 60 mg per ml of Mannitol were added and stirred to ensure complete solubilization and tonicity adjustment. The pH of the solution was adjusted to pH 7 to 8 using Sodium hydroxide and/or Hydrochloric or Acetic acid solution. Mycophenolic acid about 4.35 mg per ml was added and stirred to ensure complete solubilization. The pH of the solution was adjusted to pH 6.5 using Sodium hydroxide and/or Hydrochloric acid solution if required. The volume was made up using the remaining quantity of Water for Injection. The bulk solution was filtered through a 0.2 μm filter to get the final formulation of 1 ml.

### Example-15: Mycophenolate Sodium Ready to Use Injection

| Sr. No. | Ingredients | mg/mL | Role in Formulation |
|---|---|---|---|
| 1 | Mycophenolate Sodium | 4.65 | API |
| 2 | Dextrose monohydrate | 50.0 | Tonicity/Osmotic |
| | | | agent |
| 3 | Citric acid anhydrous | 2.0 | Buffer |
| 4 | Sodium hydroxide | Q.S. to | pH adjustment |
| | | pH 3.0 | |
| 5 | Water for injection | Q.S. to 1 mL | Vehicle |

Total Target Fill Volume of final vial with vehicle is 85 mL.

### Process for Preparation:

1. Water for injection was taken in a clean, sterile manufacturing vessel under continuous stirring;
2. 50 mg of Dextrose monohydrate was added to the vessel and mixed until completely dissolved to obtain a clear solution;
3. 2 mg of Citric acid anhydrous was added to the above solution and mixed until complete dissolution to form a buffered solution;
4. 4.65 mg of Mycophenolate sodium was added slowly to the buffered solution under stirring and mixed until a uniform solution was obtained;
5. The pH of the solution was adjusted to about 3.0 using sodium hydroxide under controlled conditions;
6. The final volume was adjusted with water for injection and the solution was mixed until homogeneity was achieved;
7. The resulting solution was filtered through a sterilizing grade membrane filter to obtain a sterile solution;
8. The sterile solution was filled into suitable glass vials and sealed with appropriate rubber stoppers and aluminum seals to obtain the ready-to-use injectable formulation.

Following is some information about Packaging material:

PRIMARY PACKAGING MATERIAL

| | |
|---|---|
| 1 | 100 mL clear glass vial |
| 2 | 20 mm Bromobutyl rubber stopper |
| 3 | 20 mm Alu. Tear off seal |

### Example-16: Related Substance Details During Stability Mycophenolate Sodium Ready to Use Injection as Per Example-15

| | Storage condition | | | | | | |
|---|---|---|---|---|---|---|---|
| | | 2-8 °C | | | 25 °C/60% RH | | |
| | Time Point | | | | | | |
| | | 3 | 6 | 12 | 3 | 6 | 12 |
| | Initial | month | month | month | month | month | month |
| Related | Result | Result | Result | Result | Result | Result | Result |
| Substances | | | | | | | |
| N-Oxide | ND | ND | 0.019% | 0.040% | ND | 0.019% | 0.023% |
| analog | | | | | | | |
| Mycophenolate | ND | ND | ND | ND | ND | ND | ND |
| Related | | | | | | | |
| Compound A | | | | | | | |
| Z-Mycophenolate | ND | ND | ND | ND | ND | ND | ND |
| Mofetil | | | | | | | |
| 1-Morphilino | ND | 0.006% | 0.025% | 0.055% | 0.146% | 0.254% | 0.345% |
| Ethoxy | | | | | | | |
| Unspecified | ND | 0.076% | 0.218% | 0.436% | 0.612% | 0.612% | 0.681% |
| impurity at | | | | | | | |
| RRT 0.39 | | | | | | | |
| Single | 0.048% | 0.055% | 0.040% | 0.137% | 0.042% | 0.129% | 0.291% |
| maximum | | | | | | | |
| unspecified | | | | | | | |
| impurity | | | | | | | |
| Total impurities | 0.160% | 0.175% | 0.366% | 0.745% | 0.915% | 1.113% | 1.414% |

Based on the above related substances data, the formulation demonstrates a controlled impurity profile under both refrigerated and accelerated storage conditions.

The inventors of the present invention analyzed the stability of the ready-to-use mycophenolate sodium injectable formulation stored at 2-8 °C and 25 °C/60% RH over a period of up to 12 months. At initial time point, total impurities were observed at about 0.160%, indicating a highly pure starting profile. Under refrigerated conditions (2-8 °C), total impurities showed a gradual increase to about 0.745% at 12 months, which remains well within acceptable limits. Under accelerated conditions (25 °C/60% RH), total impurities reached about 1.414% at 12 months, demonstrating predictable and controlled degradation behavior.

Among individual impurities, N-oxide analog remained low, with values up to about 0.040% under refrigerated conditions and about 0.023% under accelerated conditions. Mycophenolate related compound A and Z-mycophenolate mofetil were not detected at any time point, indicating absence of these degradation pathways. The impurity identified as 1-morpholino ethoxy showed a gradual increase, reaching about 0.055% at 12 months under refrigerated conditions and about 0.345% under accelerated conditions, remaining within acceptable limits.

An unspecified impurity at RRT 0.39 exhibited a time-dependent increase, reaching about 0.436% at 12 months under refrigerated conditions and about 0.681% under accelerated conditions. The single maximum unspecified impurity remained controlled, not exceeding about 0.137% under refrigerated conditions and about 0.291% under accelerated conditions.

The inventors of the present invention establish that the impurity profile remains within regulatory acceptable thresholds throughout the study period. The gradual increase in total impurities without abrupt degradation indicates chemical stability of the formulation. The absence of significant levels of critical degradation products and controlled growth of unspecified impurities confirm that the formulation is stable under both long-term and accelerated storage conditions.

Accordingly, the formulation demonstrates suitable stability characteristics for a ready-to-use injectable product, supporting its shelf life and commercial applicability.

### Example-17: Mycophenolate Sodium Ready to Use Injections

| Sr. | | Quantity (mg/mL) | | | | | | Role in |
|---|---|---|---|---|---|---|---|---|
| No. | Ingredient | F1 | F2 | F3 | F4 | F5 | F6 | formulation |
| 1 | Mycophenolate | 4.65 | 4.65 | 4.65 | 4.65 | 4.65 | 4.65 | API |
| | sodium | | | | | | | |
| 2 | Monothioglycerol | 0.1-0.3 | — | — | 0.1-0.3 | — | — | Antioxidant |
| 3 | L Cysteine | — | 0.1-0.2 | — | — | 0.1-0.2 | — | Antioxidant |
| | hydrochloride | | | | | | | |
| 4 | Ascorbic acid | — | — | 0.5-2.8 | — | — | 0.5-2.8 | Antioxidant |
| 5 | Disodium | — | — | — | 0.1 | 0.1 | 0.1 | Chelating |
| | edetate | | | | | | | agent |
| 6 | Dextrose | 40-50 | 40-50 | 40-50 | 40-50 | 40-50 | 40-50 | Osmotic |
| | monohydrate | | | | | | | agent |
| 7 | Citric acid | 1-2.5 | 1-2.5 | 1-2.5 | 1-2.5 | 1-2.5 | 1-2.5 | Buffer |
| | anhydrous | | | | | | | |
| 8 | Sodium | Q.S. | Q.S. | Q.S. | Q.S. | Q.S. | Q.S. | pH |
| | hydroxide | to pH | to pH | to pH | to pH | to pH | to pH | adjusting |
| | | 3.0 | 3.0 | 3.0 | 3.0 | 3.0 | 3.0 | agent |
| 9 | Water for | Q.S. | Q.S. | Q.S. | Q.S. | Q.S. | Q.S. | Vehicle |
| | injection | to 1 | to 1 | to 1 | to 1 | to 1 | to 1 | |
| | | mL | mL | mL | mL | mL | mL | |

### General Process for Preparation:

1. Water for injection was taken in a clean manufacturing vessel under continuous stirring;
2. Dextrose monohydrate was added to the vessel and mixed until completely dissolved to form a clear solution;
3. Citric acid anhydrous was added to the above solution and mixed until complete dissolution to obtain a buffered solution;
4. Disodium edetate, when present, was added to the buffered solution and mixed until completely dissolved;
5. An antioxidant selected from monothioglycerol, L-cysteine hydrochloride, or ascorbic acid was added, depending on the above formulation table, and mixed until uniformly dispersed;
6. Mycophenolate sodium was added slowly to the above solution under continuous stirring and mixed until a homogeneous solution was obtained;
7. The pH of the solution was adjusted to the desired range using sodium hydroxide;
8. The final volume was adjusted with water for injection and mixed until uniformity was achieved;
9. The resulting solution was filtered through a sterilizing grade membrane filter to obtain a sterile solution;
10. The sterile solution was filled into suitable sterile containers and sealed to obtain the final ready-to-use injectable formulation.

### Example-18: Mycophenolate Sodium Ready to Dilute Injections

| Sr. | | Quantity (mg/mL) | | | | | | Role in |
|---|---|---|---|---|---|---|---|---|
| No. | Ingredient | F1 | F2 | F3 | F4 | F5 | F6 | formulation |
| 1 | Mycophenolate | 26.35 | 26.35 | 26.35 | 26.35 | 26.35 | 26.35 | API |
| | sodium | | | | | | | |
| 2 | Monothioglycerol | — | 0.5-1.75 | — | — | 0.5-1.75 | — | Antioxidant |
| 3 | L Cysteine | — | — | 0.5-1.1 | — | — | 0.5-1.1 | Antioxidant |
| | hydrochloride | | | | | | | |
| 4 | Disodium | — | — | — | 0.3-0.7 | 0.3-0.7 | 0.3-0.7 | Chelating |
| | edetate | | | | | | | agent |
| 5 | Polysorbate 80 | 10-50 | 10-50 | 10-50 | 10-50 | 10-50 | 10-50 | Solubilizer |
| 6 | Citric acid | 5-14 | 5-14 | 5-14 | 5-14 | 5-14 | 5-14 | Buffer |
| | anhydrous | | | | | | | |
| 7 | Sodium | Q.S. | Q.S. | Q.S. | Q.S. | Q.S. | Q.S. | pH |
| | hydroxide | to pH | to pH | to pH | to pH | to pH | to pH | adjusting |
| | | 7.0- | 7.0- | 7.0- | 7.0- | 7.0- | 7.0- | agent |
| | | 8.0 | 8.0 | 8.0 | 8.0 | 8.0 | 8.0 | |
| 8 | Water for | Q.S. | Q.S. | Q.S. | Q.S. | Q.S. | Q.S. | Vehicle |
| | injection | to 1 | to 1 | to 1 | to 1 | to 1 | to 1 | |
| | | mL | mL | mL | mL | mL | mL | |

Ready to Dilute formulation strategies (395.25 mg/15 mL)
Fill volume: 15 mL (Each vial to be further diluted to 85 mL using 5% Dextrose solution).

### General Process for Preparation:

1. Water for injection was taken in a clean manufacturing vessel under continuous stirring;
2. Citric acid anhydrous was added to the vessel and mixed until completely dissolved to form a buffered solution;
3. Polysorbate 80 was added to the buffered solution and mixed until uniformly dispersed to facilitate solubilization;
4. Disodium edetate, when present, was added and mixed until completely dissolved;
5. An antioxidant selected from monothioglycerol or L-cysteine hydrochloride was added, depending on the above formulation table, and mixed until uniform distribution was achieved;
6. Mycophenolate sodium was added gradually to the above solution under continuous stirring and mixed until a clear and homogeneous solution was obtained;
7. The pH of the solution was adjusted to the 7.0 to 8.0 using sodium hydroxide;
8. The final volume was adjusted with water for injection and mixed until uniformity was achieved to obtain a concentrated solution;
9. The resulting solution was filtered through a sterilizing grade membrane filter to obtain a sterile formulation;
10. The sterile solution was filled into suitable sterile vials with a defined fill volume of 15 mL;
11. The filled vials were sealed with appropriate rubber stoppers and aluminum seals to obtain the ready-to-dilute injectable formulation, wherein the formulation was further diluted prior to administration to 85 mL using 5% Dextrose solution.

The invention described herein comprises in various objects and their description as mentioned above, with respect to characteristics and processes adopted. While these aspects are emphasized in the invention, any variations of the invention described above are not to be regarded as departure from the spirit and scope of the invention as described.

1. A ready-to-use parenteral pharmaceutical composition comprising:
(a) mycophenolate or a pharmaceutically acceptable salt or free acid thereof;
(b) a buffering system maintaining pH;
(c) a tonicity adjusting agent; and
(d) an aqueous vehicle;
wherein the composition is present as a sterile solution suitable for direct administration without reconstitution and exhibits controlled impurity formation during storage at 25 °C.

2. The composition as claimed in claim 1, wherein the mycophenolate is selected from mycophenolate mofetil, mycophenolate sodium, mycophenolate mofetil hydrochloride, mycophenolic acid, or combinations thereof.

3. The composition as claimed in claim 1, wherein the buffering system comprises citric acid, citrate salts, or combinations thereof, maintaining pH in the range of 2.5 to 8.0.

4. The composition as claimed in claim 1, wherein the tonicity adjusting agent is selected from sodium chloride, dextrose, mannitol, or combinations thereof, providing isotonicity suitable for parenteral administration.

5. The composition as claimed in claim 1, further comprising a solubilizer selected from polysorbate 80, polysorbate 20, polyoxyl castor oil, or combinations thereof, wherein the solubilizer maintains the active ingredient in a dissolved state.

6. The composition as claimed in claim 1, further comprising an antioxidant selected from monothioglycerol, L-cysteine hydrochloride, ascorbic acid, or combinations thereof, wherein the antioxidant reduces oxidative degradation during storage.

7. The composition as claimed in claim 1, further comprising a chelating agent selected from disodium edetate, wherein the chelating agent reduces metal ion-induced degradation.

8. The composition as claimed in claim 1, wherein the composition exhibits a controlled impurity profile characterized in that:
(i) total impurities remain below about 1.5% after storage for 12 months under accelerated conditions of about 25 °C and 60% relative humidity; and
(ii) individual impurities increase in a gradual and predictable manner without formation of any new major degradation product.

9. A ready-to-dilute sterile parenteral pharmaceutical composition comprising:
(a) mycophenolate or a pharmaceutically acceptable salt or free acid thereof in a in a concentrated form;
(b) a buffering system;
(c) a solubilizer; and
(d) an aqueous vehicle;
wherein the composition is provided as a concentrated solution requiring dilution with a pharmaceutically acceptable diluent prior to administration.

10. A pharmaceutical product comprising:
(a) the composition as claimed in claim 1; and
(b) a sterile container selected from a glass vial, infusion bag, or prefilled container system;
wherein the composition is maintained in a ready-to-use form without requirement of reconstitution prior to administration.

11. The pharmaceutical product of claim 10, wherein the mycophenolate is selected from mycophenolate mofetil, mycophenolate sodium, mycophenolate mofetil hydrochloride, mycophenolic acid, or combinations thereof.

12. The pharmaceutical product of claim 10, wherein the buffering system comprises citric acid, citrate salts, or combinations thereof, maintaining pH in the range of 2.5 to 8.0.

13. The pharmaceutical product of claim 10, wherein the tonicity adjusting agent is selected from sodium chloride, dextrose, mannitol, or combinations thereof, providing isotonicity suitable for parenteral administration.

14. The pharmaceutical product of claim 10, further comprising a solubilizer selected from polysorbate 80, polysorbate 20, polyoxyl castor oil, or combinations thereof, wherein the solubilizer maintains the active ingredient in a dissolved state.

15. The pharmaceutical product of claim 10, further comprising an antioxidant selected from monothioglycerol, L-cysteine hydrochloride, ascorbic acid, or combinations thereof, wherein the antioxidant reduces oxidative degradation during storage.

16. The pharmaceutical product of claim 10, further comprising a chelating agent selected from disodium edetate, wherein the chelating agent reduces metal ion-induced degradation.

17. The pharmaceutical product of claim 10, wherein the composition exhibits a controlled impurity profile characterized in that:
(i) total impurities remain below about 1.5% after storage for 12 months under accelerated conditions of about 25 °C and 60% relative humidity; and
(ii) individual impurities increase in a gradual and predictable manner without formation of any new major degradation product.
